(19) United States
(12) Patent Application Publication
(10) Pub. No.: US 2010/0094149 A1
(43) Pub. Date: Apr. 15, 2010

(54) Noninvasive Ultrasound Cardiac Pacemaker and Defibrillator

(75) Inventors: Andrew R. Kohut, Philadelphia, PA (US); Peter B. Kurnik, Moorestown, NJ (US); Peter A. Lewin, Wyndmoor, PA (US); Christopher Vecchio, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Philadelphia Health & Education Corporation, d/b/a Drexel University College of Medicine, Philadelphia, PA (US)

(21) Appl. No.: 12/558,294

(22) Filed: Sep. 11, 2009

Related U.S. Application Data
(60) Provisional application No. 61/096,564, filed on Sep. 12, 2008.

(51) Int. Cl. A61B 5/0472; A61N 7/00

(52) U.S. Cl. 600/509; 601/2

(57) ABSTRACT

A device for providing extracorporeal cardiac pacing. The device includes an ultrasound transducer mountable to the external thorax of a patient and an ultrasound generator for transmitting ultrasound pulses to the ultrasound transducer. The heart rate of a patient is monitored by the device. A controller evaluates the heartbeat as compared with threshold criteria for stimulation of the heart and causes the ultrasound generator to deliver ultrasound pulses to the ultrasound transducer at a prescribed intensity, frequency, and pulse duration.

### CROSS-REFERENCE TO RELATED APPLICATIONS

This application claims the priority of U.S. Provisional Application No. 61/096,564 filed on Sep. 12, 2008, which is incorporated herein by reference herein in its entirety.

### BACKGROUND OF THE INVENTION

Cardiac pacemakers have been in existence for almost 50 years. In 2005, nearly 300,000 patients received permanent pacemakers (PPM) or implantable cardioverter-defibrillators (ICD) to prevent fainting, improve symptoms associated with slow heart rates, and prevent sudden death.

There are two categories of cardiac pacemakers, temporary and permanent. The most recent statistics indicate that about 1.3 million temporary cardiac pacemakers are used annually in the US at a total cost of about $3.4 billion dollars. Globally the figures are much higher.

Two techniques for temporary cardiac pacing are currently in widespread use. The first technique employs surgically implanted intravascular catheters and leads which extend to the heart muscle. The second technique uses transcutaneous electrical pacing, which is painful and impractical for prolonged utility. These approaches have not significantly changed in the last 40 years and often result in myriad complications that increase morbidity and mortality, and are also very costly. Complications can include infection, drug toxicity, renal failure due to prolonged use of antibiotics, and death. Therefore, improved devices and techniques are needed to generate better clinical outcomes, to reduce medical complications, and to reduce costs.

Both a PPM device and a ICD device require a surgical procedure to implant and make the device operative. Therefore, such devices create potential sites for infection, particularly during implantation or removal of the device. In particular, a current pacemaker such as a PPM or an ICD requires subcutaneous surgical implantation of an electrical signal or impulse generator. Further, a current pacemaker requires surgical implantation of electrical leads, which are passed through the blood vessels and are imbedded in the wall of the heart. All surgical procedures carry some inherent risk. Moreover, once a current pacemaker device is implanted, the risk is not ended, because there exists an ongoing risk associated with device infections, device-related complications requiring extraction or access to the device, and vascular rupture.

The reported incidence of cardiac device infections ranges from 0.13% to 19.9%. Infected devices and leads must be surgically removed, and the complication rate of lead extractions is 2.0 to 2.5%. The average economic cost per treatment has been estimated at $25,000 for PPM infection and $50,000 for ICD infection. After lead extraction, temporary pacing of the heart is commonly required.

Accordingly, it would be advantageous to provide a device for cardiac pacing and defibrillation that does not require surgical implantation and thus greatly reduces the risk of infection and surgical complications.

### SUMMARY

A device is provided for extracorporeal cardiac pacing. The device includes an ultrasound transducer mountable to the external thorax of a patient, an ultrasound generator for transmitting ultrasound pulses to the ultrasound transducer, and an apparatus for monitoring the heartbeat of a patient. A controller evaluates the heartbeat as compared with threshold criteria for stimulation of the heart and causes the ultrasound generator to deliver ultrasound pulses to the ultrasound transducer at a prescribed intensity, frequency, and pulse duration.

A method is provided for cardiac pacing using an ultrasound device including an ultrasound transducer. The method includes disposing the ultrasound transducer in contact with the external thorax of a patient and causing the ultrasound transducer to deliver ultrasound pulses at a frequency in the range of about 50 kHz to about 5 MHz.

### BRIEF DESCRIPTION OF THE FIGURES

FIG. 1 is a perspective view showing an embodiment of a noninvasive ultrasound cardiac pacemaker/defibrillator device including an adhesive patch transducer, control electronics, and electro-cardiogram electrodes.

FIG. 2 is a schematic view showing an embodiment of a noninvasive ultrasound cardiac pacemaker/defibrillator device being applied to a pig during animal testing, the device including an electro-cardiogram monitor and an ultrasound generator so that ultrasound bursts can be synchronized with the underlying cardiac rhythm.

FIG. 3 is a graph of a typical electro-cardiogram waveform showing the timing of the application of ultrasound pulses for pacemaking, with the ultrasound pulses being delivered after the T-wave and before the QRS complex, thus avoiding the dangerous arrhythmia-susceptible period of the cardiac cycle.

### DETAILED DESCRIPTION

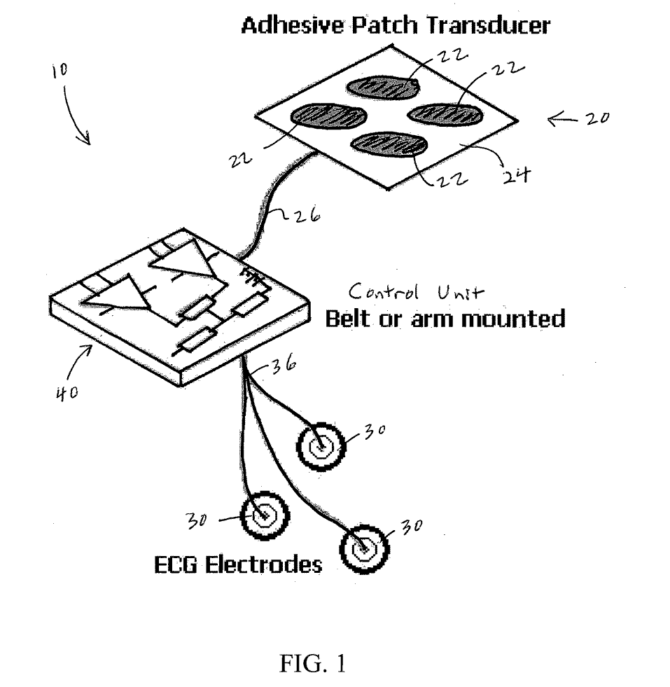

An embodiment of a noninvasive ultrasound cardiac pacing device 10 according to the present invention is shown in FIG. 1. As depicted, the device 10 comprises an ultrasound transducer 20, a set of ECG electrodes 30, and a control unit 40.

The transducer 20 includes one or more ultrasound transducing elements 22 for transmitting ultrasound to a patient. As shown, the transducing elements 22 are mounted on an adhesive patch 24 to facilitate securing of the transducer 20 to the skin of a patient. The transducer 20 is placed external to the thorax of the patient, such as on the chest wall or on the rib cage along the back of a patient, externally to the body. The location of the transducer 20 can be optimized for a particular patient by, for example, disposing one or more transducer elements 22 in the space between the ribs or by providing multiple adhesive patches 24, each mounting one or more transducer elements 22, so that the transducer elements 22 may be spaced apart from each other to best direct ultrasound into the chest cavity toward the heart muscles to be stimulated.

The transducer elements 22 are oriented to provide ultrasound that is sufficient and targeted to be able to initiate myocardial depolarization and to thus establish a pacing focus. Because all of the transducer elements 22, as well as the ECG electrodes 30 and the control unit 40, are located extracorporeally, the ultrasound cardiac device 10 can be deployed in a manner that is truly noninvasive. To deploy the device 10 on a patient, no surgical or catheterization procedures, implanted receivers, or intravascular leads are required. Thus, the device 10 could dramatically reduce device-related complications, including vascular rupture, infection, and need for extraction.

In one embodiment, the transducer elements 22 are each capable of outputting ultrasound pulses in a frequency range from about 50 kHz to about 5 MHz. Alternatively, transducer elements 22 can be used that are adapted to generate high energy pulses at much narrower frequency bands within that wide range that prove to be particularly effective during testing at penetrating the intervening tissue between the chest wall and the heart muscle while transmitting a therapeutically effective dose of ultrasound waves to pace or stimulate the heart muscle.

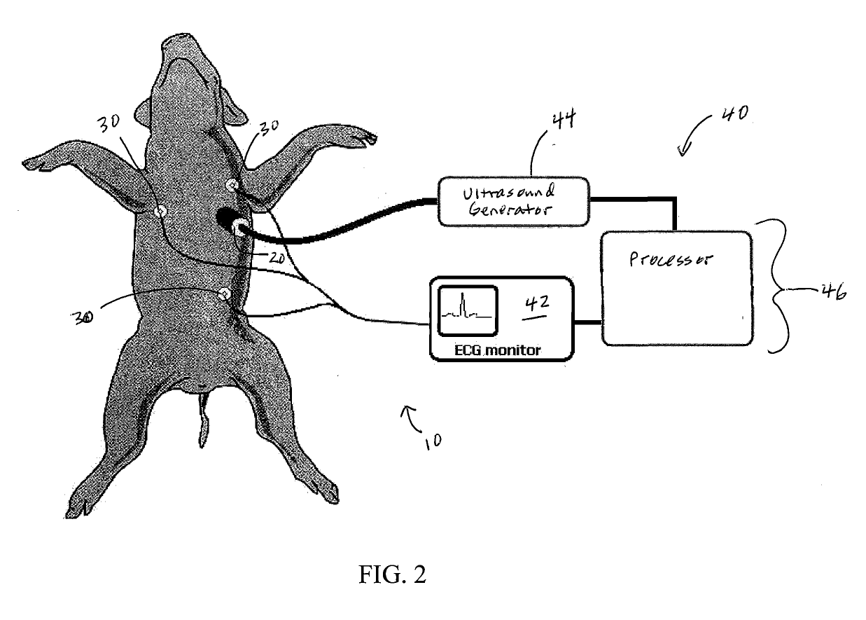

FIG. 2 depicts an embodiment of the device 10 deployed on a porcine test subject. A porcine model is preferred for early testing because pigs and humans share similarities in terms of the anatomical heart size, location and position of the heart in the thoracic cavity, and intrinsic heart rate. Porcine heart rhythm is monitored using surface electrodes 30 and a multi-channel electrocardiogram monitor 42. The surface electrodes 30 are placed on the chest surface with coupling gel. An A-mode ultrasound imager (not shown) is used to identify a location on the chest wall that provides access to the myocardium with a minimum of obstructions. In early testing, the external ultrasound pulses are planned to be synchronized with the native heart rhythm using an electrocardiogram to avoid inducing ventricular arrhythmia.

In the depicted embodiment, a set of three ECG electrodes 30 is used to monitor the patient's heartbeat, and one transducer element 22 is deployed as the ultrasound transducer 20 external to the patient's chest in the vicinity of the heart. However, it is understood that other apparatuses known in the art can be used to monitor heart rate, including but not limited to a pulse detector. The set of ECG electrodes 30 is used in a manner known in the art to monitor the heartbeat of a patient and to determine whether pacing and/or defibrillation of the heart is necessary.

The control unit 40 includes the ECG monitor 42, an ultrasound generator 44, and a control processor 46. For convenience, the control unit 40 can be mounted to a belt or armband worn by the patient, so that the patient can be fully ambulatory and mobile. The ECG electrodes 30 transmit signals via conductors 46 to the ECG monitor 42 of the control unit 40, which processes the signals transmitted from the ECG electrodes 30 communicates the ECG signal information to the processor 46. The processor 46 evaluates the status of the patient's heartbeat as compared with threshold criteria to determine whether pacing and/or defibrillation may be required. If the control processor 46 determines that pacing and/or defibrillation is required, the processor 46 provides a signal to the ultrasound generator 44, instructing the generator 44 to generate ultrasound pulses at a prescribed power level (intensity), frequency, and pulse duration. The ultrasound generator 44 includes one or more signal generators and one or more power amplifiers as required to drive the transducer elements 22. The intensity, frequency, and pulse duration, and the number, size, and spacing of transducer elements 22, can be optimized in general and for specific patients in terms of transducer matching and efficiency. In one example, a frequency in the range of about 500 kHz to about 3.5 MHz is effective, with about 1 MHz being more effective. In another example, pulse durations of about 1 millisecond to about 50 milliseconds is effective, with pulse duration of about 1 millisecond to about 3 milliseconds being more effective. In another example, intensities on the order of about 300 mW/cm² are effective. Overall, an effective exposure regimen is designed to generate between about 1 gram and about 10 grams of force in the heart muscle.

The ultrasound device 10 can initiate periodic myocardial depolarization resulting in cardiac contraction thereby establishing a consistent and reliable pacing rhythm without need for the direct or indirect application of electric energy. The ultrasound device 10 operates at intensities and frequencies that follow current FDA guidelines to provide rapidly-deployable, noninvasive, painless pacing. Accordingly, the device 10 can be employed to provide pacing for time periods spanning from minutes to weeks in conscious patients, particularly those who require temporary or emergency pacing. Temporary cardiac pacemakers often represent the first therapeutic approach for the ultimate correction of a serious cardiac problem or malady. Such situations often result from bradyarrhythmias, myocardial infarction, drug toxicity, bloodstream infection, during patient transport in emergency situations, or in the aftermath of heart surgery. Use of the noninvasive ultrasound device 10 as disclosed herein avoids the need in such patients for painful and invasive temporary intravascular pacing catheters or transcutaneous electrical pacers.

Consequently, from a position on the outside of the chest wall, the device is designed to consistently and reliably transmit ultrasound bursts from the surface of the chest wall through varied layers of tissue to induce depolarization of the cardiomyocytes. This technology can also be used to identify ideal pacing sites during atrial-ventricular and biventricular timing optimization in cardiac resynchronization therapy for a patient with heart failure. This device can be modified to provide multiple pacing foci to provide biventricular pacing and to prevent myocardial remodeling. Moreover, an array of ultrasound can be simultaneously delivered to multiple sites in the heart, providing a means of painless defibrillation. An ultrasound device according to the present invention is noninvasive and external, and provides painless and comfortable pacing and defibrillating as an alternative to the existing invasive technologies for the heart.

The ultrasound cardiac device does not create potential sites for infection when it is deployed or removed. In particular, because the ultrasound device is completely extracorporeal, it does not require surgical implantation of a generator or electrical leads or any other component; the ultrasound device transmits ultrasonic pulses through the tissues of the chest without any components that physically penetrate the skin.

It is known that mechanical stimulation can initiate pacing, a phenomenon observed each day in the cardiac catheterization lab when a catheter tip touches heart muscle and induces extra beats. The advance of the present device 10 is to provide such mechanical stimulation via ultrasound by using an ultrasound transmitter 20 that is located external to the body. Without being bound by theory, it is believed that the ultrasound can stimulate and/or stretch connective tissue such as extracellular matrix proteins, to facilitate cardiac function, or can effect the cardiovascular autonomic reflexes.

Ultrasound can interact with tissue through either thermal or non-thermal physical mechanisms. The insonification scheme used by the device 10 rules out any temperature effects (i.e., there will be no or minimal heating of the heart muscle tissue), so the device 10 operates based on non-thermal effects. In particular, the device 10 employs radiation force which can be used to stimulate cardiac and neural tissue. Extracorporeal ultrasound at FDA-approved levels can induce tissue motion at depths of up to several centimeters or more through the mechanism of acoustic radiation force. Additionally, without being bound by theory, it is believed that high-intensity ultrasound applies radiation force on the heart or neural tissue and thereby stimulates cardiac and neural tissue by activating both mechano-sensitive nonmyocytes as well as the stretch-dependent sodium ion channels of myocytes, resulting in myocytal depolarization and subsequent cardiac contraction. The use of a noninvasive, external ultrasound source, as provided by the ultrasound pacemaker device, is to initiate myocardial depolarization and thus establish a consistent and reliable pacing force without need to convert this ultrasound energy to electric energy. In particular, the device is designed to comprise part of a feasible and safe noninvasive ultrasound pacing system.

Direct cardiac pacing with ultrasound energy in an animal model has been demonstrated primarily in an attempt to seek a model for commotio cordis (a sudden disturbance of the heart resulting from a blunt, non-penetrating impact). Such direct pacing has been applied acoustic energy directly to a pig heart via midsternal thoracotomy (i.e., not transcutaneously), providing surface-to-surface contact between the ultrasound probe and the myocardium. Based upon the inventors' calculations, the radiation pressure exerted at the surface of the heart during direct pacing experiments was on the order of 16 Pascals, which corresponds to a force of approximately 3.25×10⁻² Newtons (3.25 grams) distributed over a 5 centimeter diameter contact area. Therefore, the externally disposed device 10 can be effective without opening the chest by applying increased power levels to compensate for the absorption of the additional intervening tissue structures and by applying ultrasound intercostally (i.e., between the ribs).

Following FDA guidelines, ultrasound intensity levels are preferably maintained at a spatial peak temporal average of 720 mW/cm² or less. It is believed that the device 10 can be effective at pacing and defibrillation using intensities well within this range. However, it is understood that higher ultrasound intensities may be employed, particularly for short term defibrillation, to establish a pacing focus, or in a clinical emergency situation, as required for the device 10 to effectively transmit the required ultrasound from an external location to the heart.

Circuitry in the control unit 40 senses the QRS peak and triggers a delayed burst of energy from a power amplifier and several transducers capable of high-power pulsed operation in the approximately 50 kHz to approximately 5 MHz range. Transducers can include a prefabricated transducer intended for HIFU (high intensity focused ultrasound) research, purchased from Sonic Concepts Inc., and a selection of piezoelectric transducers elements that are commercially available in ranges from about 115 kHz to about 2.5 MHz.

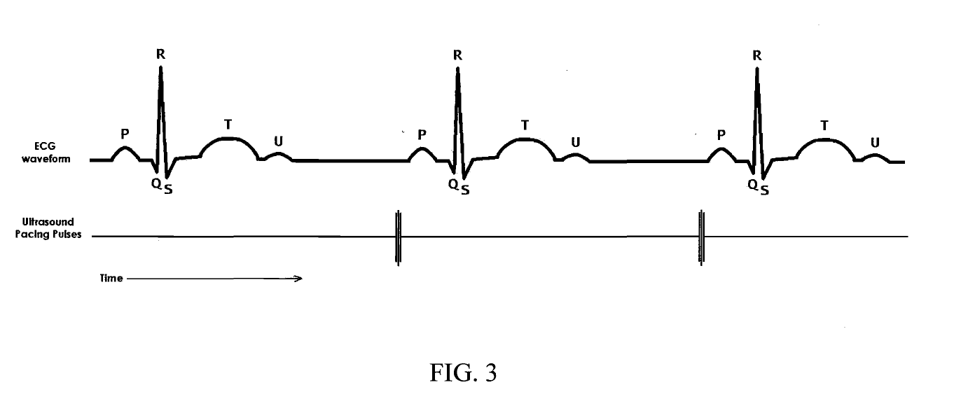

FIG. 3 depicts a typical ECG waveform with ultrasound pacing bursts applied in this manner, after the T-wave and before the QRS complex. Pulse durations of about 1 millisecond (ms) to about 50 ms will be used while targeting a sound pressure level of about 2.5 MPa (megapascal) to about 3.5 MPa at the site of stimulation. The ultrasound-initiated beats are identified by the morphology of their QRS complexes and timing relative to the ultrasound pulse. The pulses are triggered after a delay following the QRS complex and delivered in the post T-wave interval, so as to avoid the R-on-T phenomenon leading to ventricular arrhythmia.

The ultrasound device has extensive clinical applicability, since it provides noninvasive, comfortable, and reliable pacing for both temporary use (e.g., on the order of minutes) and permanent use (e.g., on the order of months or years). Deploying the ultrasound device on a patient does not require sedation or anesthesia. Specifically, the ultrasound pacemaker device can be used in conscious patients requiring temporary or emergency pacing, such as those with bradycardia or asystole in the setting of a bloodstream infection, recent myocardial infarction, or drug toxicity. Also, the ultrasound pacemaker device can provide temporary or emergency pacing or during ambulance and/or helicopter transportation. Thus, the need for painful and invasive intravascular placement of temporary pacing catheters as well as painful transcutaneous pacing can be reduced or eliminated completely by use of the ultrasound pacemaker device.

The disclosures of each and every patent, patent application, and publication cited herein are hereby incorporated herein by reference in their entirety. While the invention has been disclosed with reference to certain preferred embodiments, numerous modifications, alterations, and changes to the described embodiments are possible without departing from the sphere and scope of the invention, as defined in the appended claims and equivalents thereof. Accordingly, it is intended that the invention not be limited to the described embodiments, but that it have the full scope defined by the language of the following claims.

What is claimed is:

1. A device for providing extracorporeal cardiac pacing, comprising:
an ultrasound transducer mountable to the external thorax of a patient;
an ultrasound generator for transmitting ultrasound pulses to the ultrasound transducer;
an apparatus for monitoring the heart rate of the patient; and
a controller for evaluating the heartbeat as compared with threshold criteria for stimulation of the heart and for causing the ultrasound generator to deliver ultrasound pulses to the ultrasound transducer at a prescribed intensity, frequency, and pulse duration.

2. The device of claim 1, wherein:
the apparatus for monitoring the heart rate comprises a set of electro-cardiogram electrodes and an electro-cardiogram monitor.

3. The device of claim 1, wherein:
the apparatus for monitoring the heart rate comprises a pulse detector.

4. The device of claim 1, wherein:
the ultrasound transducer includes an ultrasound transducer element.

5. The device of claim 1, wherein:
the ultrasound transducer includes a plurality of ultrasound transducer elements.

6. The device of claim 1, wherein:
the ultrasound transducer includes at least one ultrasound transducer element and an adhesive patch for adhering the transducer element in contact with the thorax of the patient.

7. The device of claim 1, wherein:
the ultrasound generator delivers ultrasound pulses in the frequency range of about 50 KHz to about 5 MHz.

8. The device of claim 7, wherein:
the ultrasound generator delivers, ultrasound pulses in the frequency range of about 500 KHz to about 1.5 MHz.

9. The device of claim 1, wherein:
the ultrasound generator delivers ultrasound pulses having a duration of about 1 ms to about 50 ms.

10. The device of claim 1, wherein:
the ultrasound generator delivers ultrasound pulses having a duration of about 1 ms to about 3 ms.

11. The device of claim 1, wherein:
the ultrasound generator causes the ultrasound transducer to radiate ultrasound creating about 1 gram to about 10 grams of radiation force in the heart muscle.

12. The device of claim 1, wherein:
the ultrasound generator causes the ultrasound transducer to deliver ultrasound pulses at a sound pressure level of between about 2.5 MPa and about 3.5 MPa.

13. The device of claim 1, wherein:
the controller causes the ultrasound generator to deliver ultrasound pulses after the T-wave and before the QRS complex of the heart.

14. The device of claim 13, wherein:
the T-wave and the QRS complex of the heart are determined by an electro-cardiogram monitor.

15. A method of providing cardiac pacing using an ultrasound device including an ultrasound transducer, comprising:
disposing the ultrasound transducer in contact with the external thorax of a patient; and
causing the ultrasound transducer to deliver ultrasound pulses at a frequency in the range of about 50 kHz to about 5 MHz.

16. The method of claim 15, wherein the ultrasound pulses have an intensity of less than about 720 milliwatts per square centimeter.

17. The method of claim 15, further comprising:
determining the existence of a clinical emergency situation; and
causing the ultrasound transducer to deliver ultrasound pulses at an intensity in excess of about 720 milliwatts per square centimeter.

18. The method of claim 15, further comprising:
locating at least one ultrasound transducing element of the ultrasound transducer in an intercostal position.

19. The method of claim 15, wherein the ultrasound transducer delivers ultrasound pulses having a duration from about 1 ms to about 10 ms.

20. The method of claim 15, wherein the ultrasound transducer delivers ultrasound pulses timed to occur after the T-wave and before the QRS complex in the heartbeat of a patient.
